(19) United States
(12) Patent Application Publication
(10) Pub. No.: US 2024/0390605 A1
(43) Pub. Date: Nov. 28, 2024

(54) IMAGING SYSTEM AND METHOD FOR INTEGRATED VASCULAR ACCESS DEVICE INDWELL ASSESSMENT AND DATA INTEGRATION

(71) Applicant: Becton, Dickinson and Company, Franklin Lakes, NJ (US)

(72) Inventors: Jonathan Karl Burkholz, Salt Lake City, UT (US); Steffan Sowards, Salt Lake City, UT (US); Shayne Messerly, Kaysville, UT (US); Eric Lindekugel, Salt Lake City, UT (US)

(21) Appl. No.: 18/658,791

(22) Filed: May 8, 2024

Related U.S. Application Data
(60) Provisional application No. 63/468,940, filed on May 25, 2023.

(51) Int. Cl. A61M 5/42; A61M 25/01; A61M 25/02

(52) U.S. Cl. CPC A61M 5/427; A61M 25/02; A61M 2025/0166; A61M 2025/0266; A61M 2205/3375; A61M 2205/583

(57) ABSTRACT

Imaging systems and methods for integrated vascular access device indwell assessment and data integration are provided. Such imaging systems and methods can be used to better assess and monitor the status of an indwelling integrated vascular access device and the overall viability of the vascular access in the acute care and alternate site setting and to reduce the patient complications and experience, clinician burden, and overall effectiveness of the patient's treatment and care. Such imaging systems and methods can also facilitate the use of imaging devices, such as ultrasound devices, to assess the current state of an indwelling vascular access device and compare it to a prior state or established clinical standard. This may facilitate predictive detection, identification, and/or diagnosis of an emerging risk of a catheter related complication or detection of an actual complication intermittently and consistently.

### RELATED APPLICATIONS

This application claims the benefit of U.S. Provisional Patent Application No. 63/468,940, which was filed on May 25, 2023, which is incorporated herein in its entirety.

### BACKGROUND

Vascular access devices are commonly used for a variety of infusion therapies. For example, vascular access devices may be used for infusing therapeutic agents or fluids into a patient. Vascular access devices may also be used for withdrawing blood from the patient. There are a variety of vascular access devices commonly used in a medical setting, including, for example, peripherally-inserted central catheters, midline catheters, central venous catheters, dialysis catheters, and arterial catheters.

A common type of vascular access device includes a catheter that is over-the-needle. As its name implies, the catheter that is over-the-needle may be mounted over an introducer needle having a sharp distal tip. The catheter and the introducer needle may be assembled so that the distal tip of the introducer needle extends beyond the distal tip of the catheter with the bevel of the needle facing up away from skin of the patient. The catheter and introducer needle are generally inserted at a shallow angle through the skin into vasculature of the patient. To verify proper placement of the introducer needle and/or the catheter in the blood vessel, a clinician generally confirms that there is “flashback” of blood in a flashback chamber of the catheter assembly. Once placement of the needle has been confirmed, the catheter may be left in place for future blood withdrawal or fluid infusion.

Although catheter indwell performance (i.e., how long the catheter can be safely left in the vasculature) has improved in recent years, there remains a significant number of complications that may develop throughout the intended dwell time of a vascular access device. These complications may include infiltration, extravasation, dislodgement, occlusion, loss of patency, infection, catheter kinking, catheter movement, thrombus development, and phlebitis. These complications may also include localized changes in a patient's physiology, such as vein or arterial size, collapse, stiffening, damage, and other changes that may accelerate further development of complications.

The subject matter claimed herein is not limited to embodiments that solve any disadvantages or that operate only in environments such as those described above. Rather, this background is only provided to illustrate one example technology area where some implementations described herein may be practiced.

### SUMMARY

The present disclosure relates generally to imaging systems and methods for integrated vascular access device indwell assessment and data integration. Such imaging systems and methods can be used to better assess and monitor the status of an indwelling integrated vascular access device and the overall viability of the vascular access in the acute care and alternate site setting and to reduce the patient complications and experience, clinician burden, and overall effectiveness of the patient's treatment and care. Such imaging systems and methods can also facilitate the use of imaging devices, such as ultrasound devices, to assess the current state of an indwelling vascular access device and compare it to a prior state or established clinical standard. This may facilitate predictive detection, identification, and/or diagnosis of an emerging risk of a catheter related complication or detection of an actual complication intermittently and consistently.

Embodiments of the present disclosure may be implemented as a securement platform that includes a base layer, a vascular access device pocket formed in the base layer, a catheter insertion site window formed in the base layer distal to the vascular access device pocket, and an imaging device pocket.

In some embodiments, the vascular access device pocket may be shaped and sized to correspond with a stabilization platform of a vascular access device.

In some embodiments, the vascular access device pocket may be a cutout in the base layer.

In some embodiments, the vascular access device pocket may include an adhesive portion of an upper surface of the base layer.

In some embodiments, the catheter insertion site window may overlap with the vascular access device pocket.

In some embodiments, the catheter insertion site window may be spaced from the vascular access device pocket.

In some embodiments, the imaging device pocket may be formed in the base layer.

In some embodiments, the base layer may be formed in a separate component from the vascular access device pocket and the catheter insertion site window.

In some embodiments, the securement platform may include a guide that at least partially surrounds the imaging device pocket.

In some embodiments, the guide may extend above the base layer.

In some embodiments, the imaging device pocket may include a gel cap.

In some embodiments, the securement platform may include a securement dressing that is configured to be positioned overtop at least a portion of the securement platform. The securement dressing may have a transparent window that is positioned overtop the catheter insertion site window.

Embodiments of the present disclosure may be implemented as an imaging system that includes a securement platform and a base unit. The securement platform may include a base layer, a vascular access device pocket formed in the base layer, a catheter insertion site window formed in the base layer distal to the vascular access device pocket, and an imaging device pocket. The base unit may be configured to receive images from an imaging device positioned within the imaging device pocket when the imaging device pocket is positioned overtop a distal tip of a catheter that is inserted into a patient's vasculature.

In some embodiments, the base unit may include an artificial intelligence engine that is configured to detect a depth of the distal tip of the catheter from the images.

In some embodiments, the imaging system may include one or more monitoring devices for displaying display content derived from the images.

In some embodiments, the securement platform may include a securement dressing.

In some embodiments, the securement platform may include a first component that includes the vascular access device pocket and the catheter insertion site window and a second component that includes the imaging device pocket.

In some embodiments, the imaging system may include a vascular access device having a stabilization platform. The vascular access device pocket may be configured to receive the stabilization platform.

Embodiments of the present disclosure may be implemented as a method for obtaining images of a patient's vasculature. A securement platform may be positioned on a patient. The securement platform may include an imaging device pocket. The imaging device pocket can be positioned overtop a catheter that is inserted through the patient's vasculature. An imaging device can be positioned in the imaging device pocket to obtain one or more images of the catheter.

In some embodiments, the securement platform may also include a base layer, a vascular access device pocket formed in the base layer, and a catheter insertion site window formed in the base layer distal to the vascular access device pocket.

It is to be understood that both the foregoing general description and the following detailed description are examples and explanatory and are not restrictive of the invention, as claimed. It should be understood that the various embodiments are not limited to the arrangements and instrumentality illustrated in the drawings. It should also be understood that the embodiments may be combined, or that other embodiments may be utilized and that structural changes, unless so claimed, may be made without departing from the scope of the various embodiments of the present invention. The following detailed description is, therefore, not to be taken in a limiting sense.

### BRIEF DESCRIPTION OF THE SEVERAL VIEWS OF THE DRAWINGS

Example embodiments will be described and explained with additional specificity and detail through the use of the accompanying drawings in which:

FIG. 1A illustrates a prior art vascular access device that could be used in one or more embodiments of the present disclosure;

FIG. 1B illustrates a securement platform that is configured in accordance with one or more embodiments of the present disclosure;

FIG. 2 illustrates an imaging system that is configured in accordance with one or more embodiments of the present disclosure;

FIG. 3 illustrates another securement platform that is configured in accordance with one or more embodiments of the present disclosure;

FIG. 4 illustrates another securement platform that is configured in accordance with one or more embodiments of the present disclosure;

FIG. 5A illustrates another securement platform that is configured in accordance with one or more embodiments of the present disclosure;

FIG. 5B illustrates the securement platform of FIG. 5A when used with a vascular access device;

FIG. 5C illustrates the securement platform of FIG. 5A when a securement dressing is used to secure the vascular access device;

FIG. 6A is a block diagram of components of an imaging system that is configured in accordance with one or more embodiments of the present disclosure;

FIG. 6B is a flow diagram representing how embodiments of the present disclosure can be used throughout the continuity of care;

FIG. 7A is a cross-sectional view of the vasculature when an imaging system is used in accordance with one or more embodiments of the present invention;

FIGS. 7B and 7C are example images generated by the imaging system of FIG. 7A;

FIG. 8 is an example display that can be generated by an imaging system that is configured in accordance with one or more embodiments of the present disclosure; and

FIG. 9 provides an example of electronic components that a base unit or monitoring device of an imaging system may include in one or more embodiments of the present disclosure.

### DESCRIPTION OF EMBODIMENTS

In this specification and the claims, the term “continuity of care” is intended to represent the entire duration of a vascular access including pre-insertion, during insertion, indwell duration, and post-removal. A “vascular access device” should be construed as encompassing an intravenous catheter device and any other device by which a patient's vasculature may be accessed. “Vascular access data” should be construed as encompassing any data relating to the access of a patient's vasculature using a vascular access device and includes images of the patient's vasculature, characteristics of the vascular access device, information about the placement and/or removal of the vascular access device, information about events that occur during the indwell of the vascular access device, complications detected, the patient's vitals, fluid and blood flow characteristics, etc.

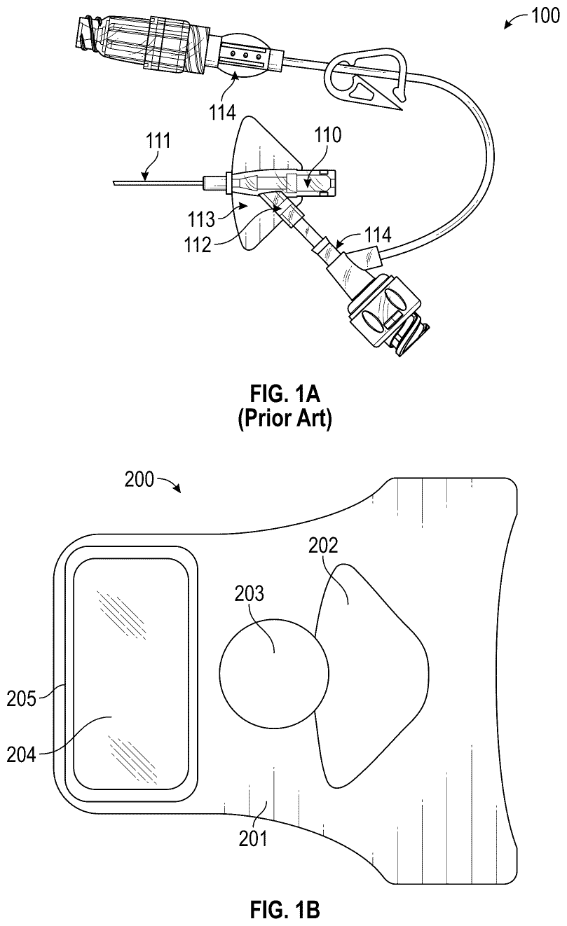

FIG. 1A provides an example of a vascular access device 100 with which an imaging system configured in accordance with embodiments of the present disclosure may be used. Vascular access device 100 includes a catheter adapter 110 from which a catheter 111 extends. Catheter adapter 111 may also include a side port 112 by which an extension set 114 is connected to catheter adapter 111. Vascular access device 100 may also include a stabilization platform 113 for stabilizing catheter adapter 111 when positioned on a patient. Vascular access device 100 is only one example of the many vascular access devices that could be used as part of embodiments of the present disclosure. For example, embodiments of the present disclosure could be used with central venous catheters (CVCs), peripherally inserted central catheters (PICCs), midline catheters, arterial catheters, peripheral intravenous catheters (PIVCs), long PIVCs, venipuncture devices, sub-cutaneous access devices, and other indwelling tube, probe, sensor, or instrument devices.

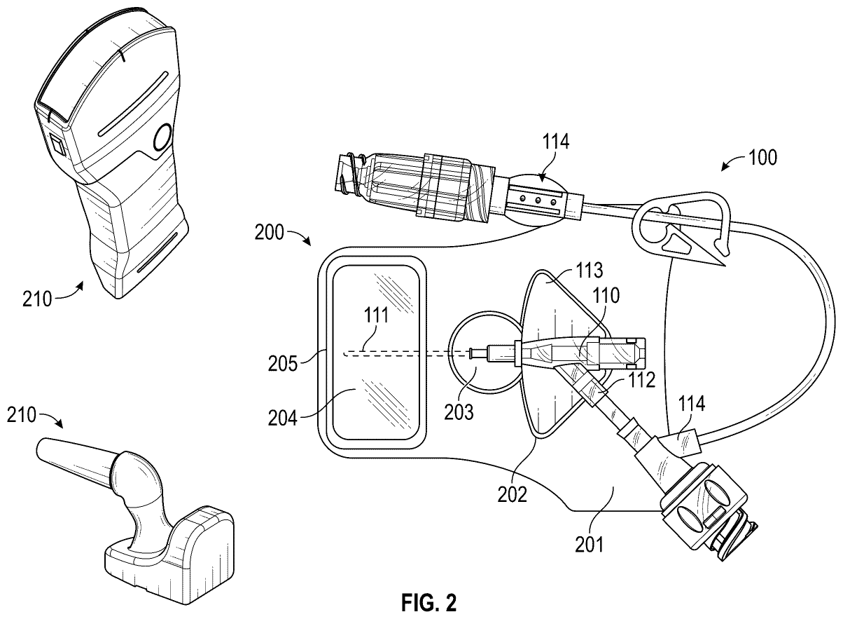

FIG. 1B provides an example of a securement platform 200 that is configured in accordance with one or more embodiments of the present disclosure. Securement platform 200 is configured for use with vascular access device 100 and can be positioned under vascular access device 100 during use as is shown in FIG. 2. Securement platform 200 includes a base layer 201 which may have an adhesive underside to allow securement platform 200 to be adhered to the patient's skin. Base layer 201 may also include a vascular access device pocket 202 that may be shaped and sized to generally match stabilization platform 113. In some embodiments, vascular access device pocket 202 may be a cutout that exposes the patient's skin thereby allowing stabilization platform 113 to be placed directly on the skin. In other embodiments, vascular access device pocket 202 may be a portion of base layer 201 having an adhesive on its upper surface so that stabilization platform 113 can be adhered to base layer 201.

Securement platform 200 can also include a catheter insertion site window 203 that is positioned distal to vascular access device pocket 202 to allow catheter 111 to pass through securement platform 200 and into the patient's vasculature when catheter adapter 110 is positioned above securement platform 200. In some embodiments, catheter insertion site window 203 may be shaped and sized to accommodate antimicrobial patches or pads or to enable the application and containment of skin adhesive for sealing the insertion site. In some embodiments, a slot (not shown) may be formed in base layer 201 and may extend between catheter insertion site window 203 and the periphery of base layer 201 to enable securement platform 200 to be placed under vascular access device 100 after insertion of catheter 111.

Securement platform 200 also includes an imaging device pocket 204 that is positioned distal to catheter insertion site window 203. Imaging device pocket 204 can be spaced from catheter insertion site window 203 at a distance that corresponds to the length of catheter 111. In other words, imaging device pocket 204 can be positioned so that it will be overtop the distal tip of catheter 111 when catheter 111 is inserted into the vasculature and stabilization platform 113 is positioned in vascular access device pocket 202. In some embodiments, imaging device pocket 204 may be a cutout that exposes the patient's skin. In other embodiments, imaging device pocket 204 may be formed of a gel cap to facilitate imaging. The size and shape of imaging device pocket 204 can be selected to accommodate a range of imaging device head shapes and orientations including rectangular, square, or other shape running in the transverse and/or longitudinal orientation. FIG. 2 shows two examples of imaging devices 210 that could be used.

In some embodiments, imaging device pocket 204 may be at least partially surrounded by a guide 205. In some embodiments, guide 205 may be raised from base layer 201 to form a wall around imaging device pocket 204. Guide 205 may facilitate controlled adjustments to an imaging device 210 when positioned in imaging device pocket 204 such as to control probe angle or rotation across multiple degrees of freedom (e.g., of an ultrasound probe).

In some embodiments where imaging device pocket 204 includes a gel cap, the gel cap may be a single-use stand-alone device that is integrated into the securement platform 200. In other embodiments, the gel cap could be attached to the patient or imaging device 210 to be readily accessible for use within imaging device pocket 204. In some embodiments, the gel cap could have antimicrobial properties to allow imaging device pocket 204 to remain clean through multiple uses of an imaging device 210. In some embodiments, a gel cap could be configured to be capable of being rehydrated so that the gel cap may be used multiple times within imaging device pocket 204.

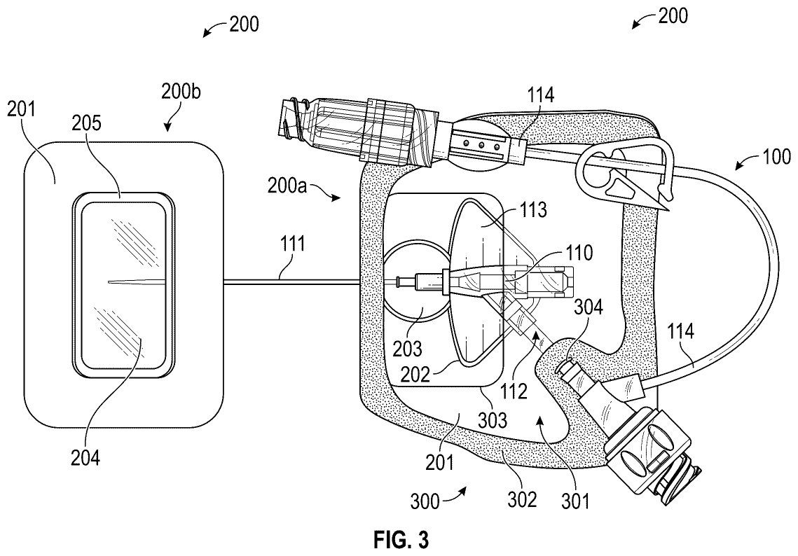

FIG. 3 provides an example where securement platform 200 is formed of two separate components 200a and 200b. Component 200a includes vascular access device pocket 202 and catheter insertion site window 203, while component 200b includes imaging device pocket 204. This two-component configuration of securement platform 200 can be used to accommodate catheters 111 of different lengths such as for long peripheral intravenous catheters and midline catheters. In some embodiments, component 200b can have an expanded base layer 201 to facilitate adhering component 200b to the patient's skin.

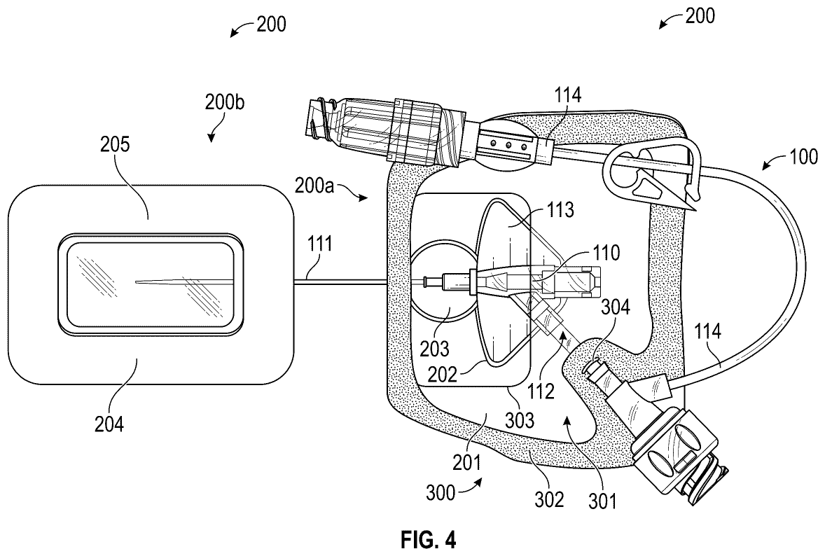

FIG. 3 also provides an example where securement platform 200 includes a securement dressing 300 that is placed overtop securement platform 200 (which in this case is overtop component 200a) to secure vascular access device 100 in place relative to securement platform 200. Securement dressing 300 can include a layer 301 that may be shaped and sized to match the proximal end of securement platform 200 (e.g., to match the size and shape of component 200a). Layer 301 may include a transparent window 303 that aligns/overlaps with catheter insertion site window 203 to facilitate viewing the insertion site. In some embodiments, transparent window 303 may align/overlap with at least a portion of vascular access device pocket 202 to facilitate viewing catheter adapter 110. Layer 301 may also include a border 302. In some embodiments, the underside of border 302 may include an adhesive for securing securement dressing 300 to securement platform 200. In some embodiments, a slot 304 may be formed in border 302 to allow extension set 114 to pass through securement dressing 300. FIG. 4 is the same as FIG. 3 but shows that component 200b can be oriented in a longitudinal orientation relative to catheter 111.

In some embodiments, securement platform 200 may consist only of component 200b. In such embodiments, securement platform 200 may be positioned appropriately to ensure that imaging device pocket 204 is overtop the distal tip of catheter 111.

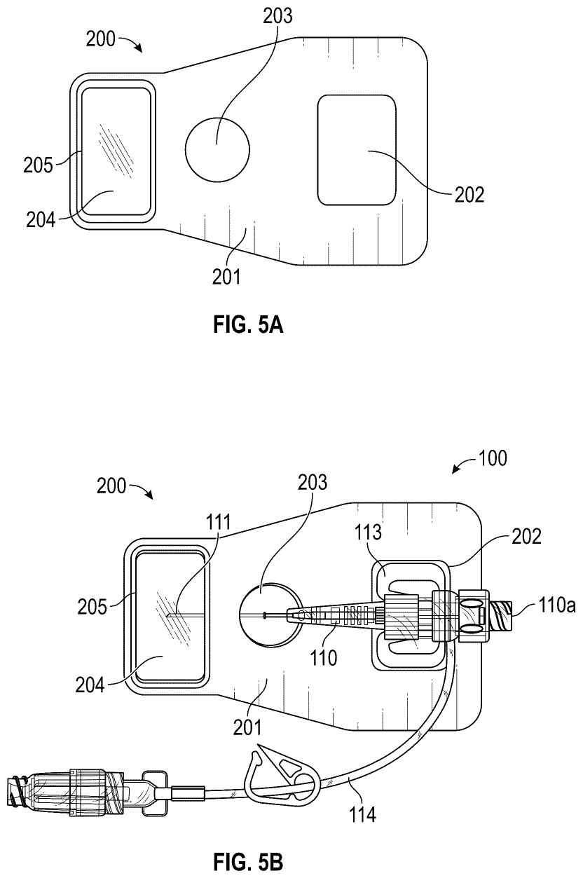

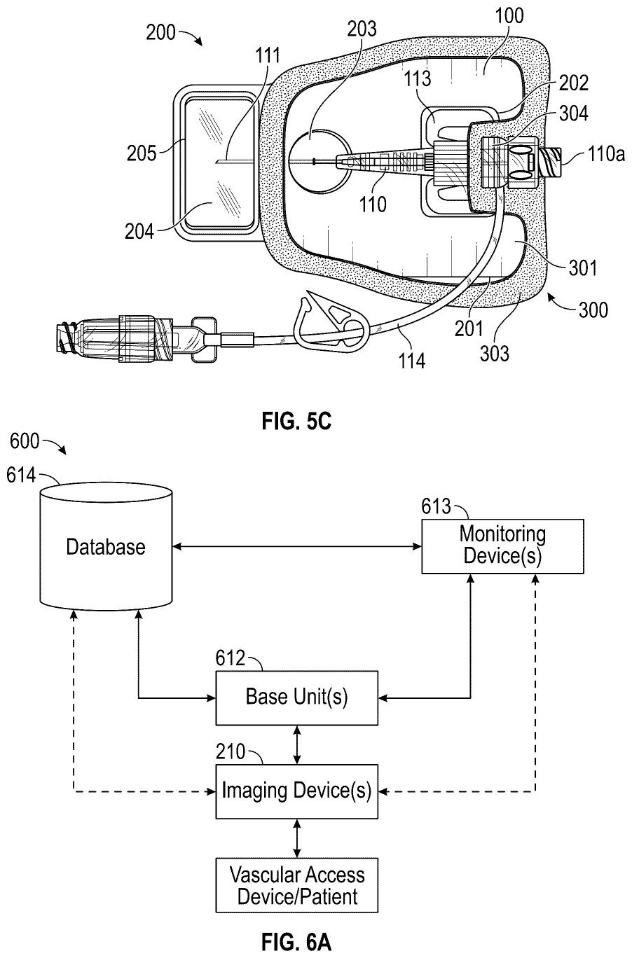

FIGS. 5A-5C provide another example of a securement platform 200 that is configured for use with a differently configured vascular access device 100, which is a non-integrated vascular access device. These figures represent how the size, shape, and relative positions of vascular access device pocket 202, catheter insertion site window 203 and imaging device pocket 204 of securement platform 200 and of transparent window 303 and slot 304 of securement dressing 300 can be configured to accommodate different vascular access devices.

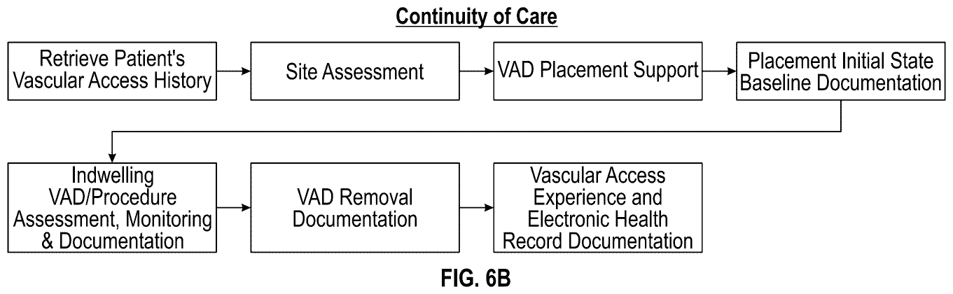

FIG. 6A provides an example of an imaging system 600 configured in accordance with one or more embodiments of the present disclosure. Imaging system 600 includes one or more imaging devices 210, one or more base units 612, one or more monitoring devices 613 and a database 614. Each imaging device 210 can be used to capture images (e.g., via ultrasound, near-infrared, optical florescence, optical reflectivity, LiDAR, or other modality) and possibly other vascular access data in connection with a vascular access device being placed in the vascular of a patient. In some embodiments, imaging system 600 could include one or more doppler devices that may be used to capture flow characteristics. A doppler device could be used in place of or in addition to an imaging device to provide functionality as described below.

A base unit 612, which may be integrated into another component of imaging system 600 in some embodiments, can represent a networking-capable computing device that is configured to communicate with database 614 and possibly with monitoring device(s) 613. For example, in some embodiments, imaging device 210 may interface directly with base unit 612 (e.g., via Bluetooth or another short-range communication protocol) for communicating vascular access data which in turn may communicate with database 614 for storing such vascular access data and/or with monitoring device(s) 613 for displaying such vascular access data. In other embodiments, imaging device 210 may have such networking capabilities and may therefore be viewed as including base unit 612.

A monitoring device 613 can be any computing device that is configured to display data related to the continuity of care. For example, a monitoring device 613 could be a personal computer, smart phone, dedicated computing device/display, etc. on which a web-based interface or dedicated application is used to display vascular access data pertaining to the continuity of care for a patient. Such monitoring devices 613 could be positioned in the patient's room or at a nursing station, carried by a clinician, etc. In some embodiments, a monitoring device 613 may include a base unit 612. For example, a monitoring device 613 could be placed next to a patient and could implement the functionality of a base unit 612 to interface with an imaging device 210 and database 614.

Database 614 is intended to represent any arrangement of computing components that may be used to store vascular access data for one or more patients. For example, database 614 could be a dedicated server computing device or cloud storage that is configured to implement database functionality.

FIG. 6B is a flow diagram representing the continuity of care throughout which embodiments enable the capture and connecting of vascular access data. The continuity of care can encompass connecting a patient's vascular access history. In other words, vascular access data pertaining to previous vascular accesses can be retrieved to connect such data throughout the continuity of care of a subsequent vascular access. The continuity of care can also include collecting and/or connecting vascular access data during a site assessment and vascular access device placement support. These stages may entail using one or more imaging devices 210 to examine the location of the patient's veins both prior to and during the placement of a vascular access device such as to identify and select the best vein for placement and to determine the appropriate catheter gauge size and length for the target vein. The one or more imaging devices 210 can be used to generate and/or present vascular access data during these two stages of the continuity of care. The continuity of care can further include collecting vascular access data in the form of placement initial state baseline documentation. This documentation may include a position of the vascular access device within the patient's vasculature, the extent to which the vascular access device is inserted into the patient's vasculature, etc. The continuity of care may also include collecting vascular access data throughout the indwell of the vascular access device such as documentation representing an assessment or monitoring of the patient and/or the vascular access device including during procedures, events, or other occurrences. Embodiments of the present disclosure may primarily be beneficial for this stage. The continuity of care may additionally include collecting vascular access data constituting documentation of the removal of the vascular access device. Finally, the continuity of care may include collecting vascular access data in the form of vascular access experience and electronic health record documentation (e.g., feedback from the patient and/or one or more clinician's that were involved in the vascular access).

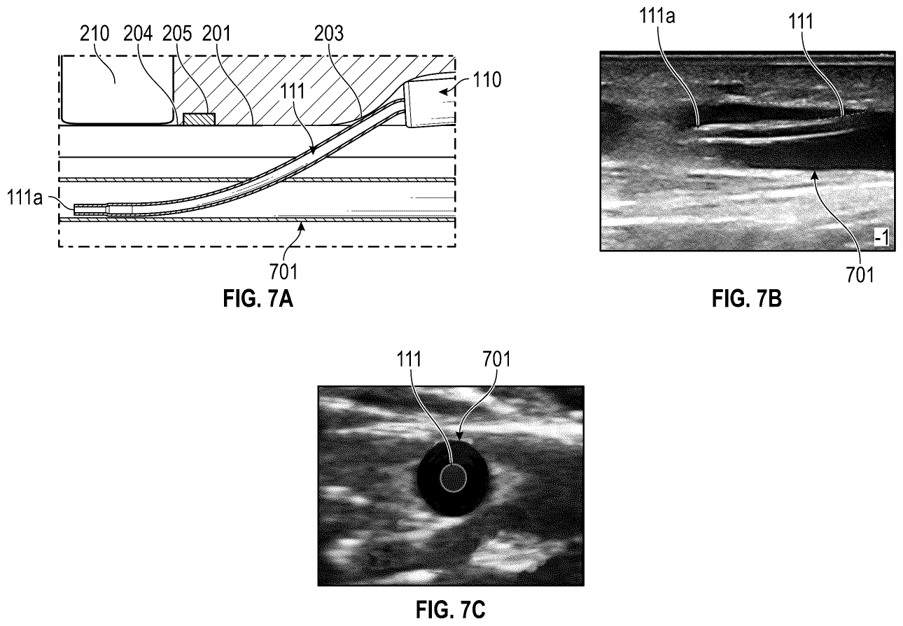

FIG. 7A is a partial cross-sectional view of a patient's vasculature 701 when securement platform 200 is used. As shown, imaging device pocket 204 is positioned overtop the distal tip 111a of catheter 111. Accordingly, a clinician can place the head of an imaging device 210 within imaging device pocket 204 to capture images of distal tip 111a. For example, FIG. 7B is an image that captures a transverse view of vasculature 701, catheter 111, and distal tip 111a, and FIG. 7C is an image that captures a cross-sectional view of vasculature 701 and catheter 111. Guide 205 can facilitate positioning imaging device 210 appropriately to capture such views clearly.

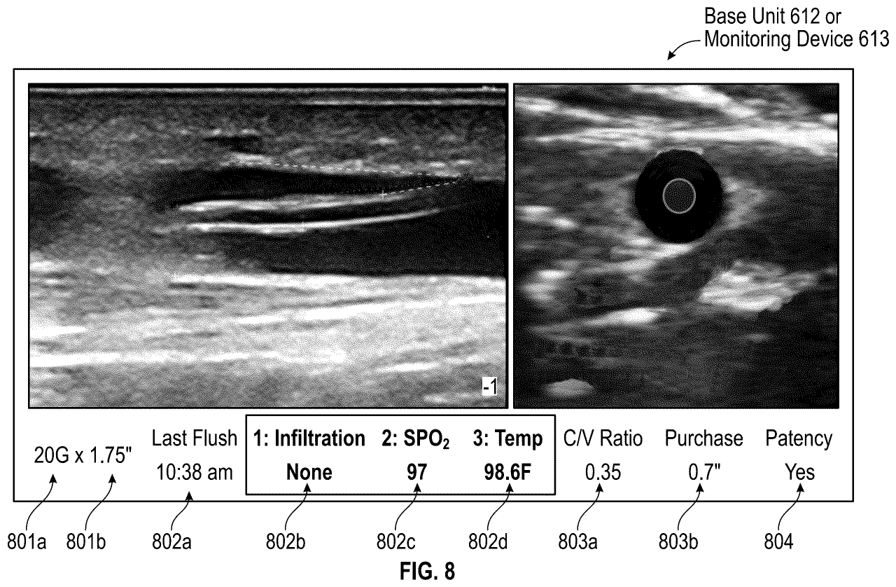

FIG. 8 provides an example of how images generated by imaging device 210 can be integrated into a display along with various information derived from the images. As indicated, this display could be generated and/or presented on base unit 612 and/or any number of monitoring devices 613. This display may include one or more views of catheter 111 within vasculature 701 such as the transverse view of FIG. 7B and the cross-sectional view of FIG. 7C. The transverse view may allow a clinician to see how catheter 111 is extending into vasculature 701 and may therefore facilitate quickly determining if catheter 111 is inserted sufficiently, if distal tip 111a is positioned correctly, if there is any blockage, or any other condition that is capable of being detected via ultrasound or other modality. The cross-sectional view may allow a clinician to see how a particular portion of catheter 111 is positioned within vasculature 701 and may therefore facilitate quickly determining if catheter 111 may be excessively limiting blood flow through vasculature 701 or any other condition that is capable of being detected via ultrasound. In some embodiments, the size and shape of imaging device window 204 can enable a user to adjust the location of the views generated by imaging device 210. For example, a user may be able to move the cross-sectional view along the length of catheter 111 to determine if there is excessive blockage at any portion along the length of catheter 111 by sliding imaging device 210 within imaging device window 204.

FIG. 8 also illustrates that the display may include a variety of vascular access data that may be derived from the images that imaging device 210 produces or from input. For example, the display includes an indicator 801a of the gauge of catheter 111 and an indicator 801b of the length of catheter 111. Indicators 801a and 801b could be obtained via user input or could be calculated from the images produced by imaging device 210.

The display also includes indicators 802a, 802b, and 802c for different parameters. In some embodiments, these parameters could be selectable. For example, in FIG. 8, indicator 802a provides information for when catheter 111 was last flushed. This last flush information could be calculated using the images produced by imaging device 210. For example, doppler techniques could be applied to the image data to detect when fluid is flowing out through distal tip 111a, and in response to such a detection, base unit 612 (or a monitoring device 613) could automatically store an indication that a flush has occurred at that time. In FIG. 8, indicators 802b and 802c have not been selected. However, these indicators and additional indicators could be selected to display information for any of many different conditions, events, statuses, etc. as described below.

The display further includes indicators 803a and 803b that provide information about the portion of catheter 111 that is inside vasculature 701. Indicator 803a defines the catheter to vein ratio (i.e., the ratio of the catheter's diameter to the vein's diameter at a particular location). Indicator 803b defines the purchase of catheter 111 (i.e., the length of catheter 111 that is inside vasculature 701 or the percentage of the catheter length that is inside the vasculature). The display additionally includes an indicator 804 defining a patency status of catheter 111 (i.e., whether catheter 111 can safely remain within vasculature 701). Base unit 612 (or a monitoring device 613) could calculate the patency status using the images provided by imaging device 210 (e.g., to detect the extent to which catheter 111 and/or vasculature 701 around catheter 111 may be blocked).

As suggested above, imaging system 600 can be configured to monitor and/or display information relating to the status of catheter 111, vasculature 701, or the surrounding tissue and a variety of associated physiological or procedural parameters by leveraging images that are provided by imaging device 210. This information includes catheter geometry information (e.g., the catheter to vein ratio, the purchase of the catheter, flow restrictions around the catheter), catheter position information (axial position of the catheter within the vein, the position or angle of the distal tip of the catheter relative a vein wall, valve, branch or other physiological feature), catheter movement or displacement, catheter kinking, dislodgment events, extravasation, infiltration detection (e.g., by monitoring tissue surrounding vasculature 501), thrombus development, phlebitis (visual or correlated cumulative movement), patency indicators, blood flow characteristics (e.g., by using doppler to detect velocity and/or volume of blood flowing into catheter 102), fluid administration flow characteristics (e.g., by using doppler to detect velocity, volume, direction, and/or duration of fluid flow), procedural events (e.g., flush, draw, fluid administration), and/or line draw tubing, probe or sensor position in the vein or relative to the distal tip of the catheter or physiological feature (e.g., thrombus, valve, wall, branch, etc.).

Imaging system 600 may provide a display including indicators of any of the above-mentioned information and may provide corresponding alerts. For example, base unit 612 or a monitoring device 613 may be configured to output a visual, audible, tactile, or digital alert when a condition or event is detected from the ultrasound images.

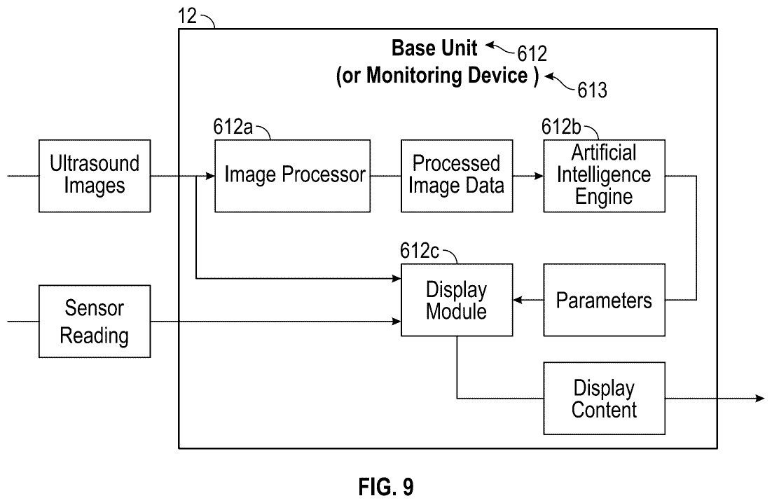

FIG. 9 provides an example of how base unit 612 (or possibly monitoring device 613) could be configured to generate display content from images generated by an imaging device 210. This display content can include any of the above-described information, indicators, status, events, alerts, etc. (collectively “parameters”). FIG. 8 is one example of display content.

Base unit 612 may be configured to receive images from imaging device 210 continuously, periodically, on demand, etc. Base unit 612 may include an image processor 612a that is configured to process the images to generate processed image data. This processed image data can be input to an artificial intelligence engine 612b that may be configured to detect and/or generate parameters from the processed image data. The parameters along with the images can be provided to a display module 612c that can generate the display content that includes the images and the parameters.

In some embodiments, image processor 612a can be configured to determine from an image or sequence of images various status information such as catheter geometry or position information or the presence of a thrombus, kink, or other blockage. In some embodiments, artificial intelligence engine 612b can be trained to detect when parameters are present in a stream of images. For example, artificial intelligence engine 612b could detect when a sequence of images is indicative of a flush event, a draw event, the occurrence of extravasation, a dislodgement or movement event, etc. In some embodiments, artificial intelligence engine 612b could be used to predict the development or increasing risk of a potential complication or event. For example, artificial intelligence engine 612b could process images to detect that the catheter purchase is changing or decreasing over time. If this trend is detected or a threshold purchase is reached (e.g., when less than some percentage of catheter length remains in the vein), artificial intelligence engine 612b could cause an alert to be triggered so that a clinician can prevent failure of the catheter.

In some embodiments, artificial intelligence engine 612b (or another artificial intelligence solution) could be used to automatically detect a depth of distal tip 111a from images generated by imaging device(s) 210. The detected depth could then be used to enhance the accuracy of the images. For example, to facilitate the use of C-mode ultrasound, imaging device 210 could generate images at preset depths and then artificial intelligence engine 612b could evaluate the images to identify which image(s) includes catheter 111. The known depth of the identified image(s) could then be used as the depth for generating further C-mode ultrasound images.

All examples and conditional language recited herein are intended for pedagogical objects to aid the reader in understanding the invention and the concepts contributed by the inventor to furthering the art and are to be construed as being without limitation to such specifically recited examples and conditions. Although embodiments of the present inventions have been described in detail, it should be understood that the various changes, substitutions, and alterations could be made hereto without departing from the spirit and scope of the invention.

What is claimed:

1. A securement platform comprising:
a base layer;
a vascular access device pocket formed in the base layer;
a catheter insertion site window formed in the base layer distal to the vascular access device pocket; and
an imaging device pocket.

2. The securement platform of claim 1, wherein the vascular access device pocket is shaped and sized to correspond with a stabilization platform of a vascular access device.

3. The securement platform of claim 1, wherein the vascular access device pocket is a cutout in the base layer.

4. The securement platform of claim 1, wherein the vascular access device pocket comprises an adhesive portion of an upper surface of the base layer.

5. The securement platform of claim 1, wherein the catheter insertion site window overlaps with the vascular access device pocket.

6. The securement platform of claim 1, wherein the catheter insertion site window is spaced from the vascular access device pocket.

7. The securement platform of claim 1, wherein the imaging device pocket is formed in the base layer.

8. The securement platform of claim 7, wherein the base layer is formed in a separate component from the vascular access device pocket and the catheter insertion site window.

9. The securement platform of claim 1, further comprising:
a guide that at least partially surrounds the imaging device pocket.

10. The securement platform of claim 9, wherein the guide extends above the base layer.

11. The securement platform of claim 1, wherein the imaging device pocket includes a gel cap.

12. The securement platform of claim 1, further comprising:
a securement dressing that is configured to be positioned overtop at least a portion of the securement platform, the securement dressing having a transparent window that is positioned overtop the catheter insertion site window.

13. An imaging system comprising:
an ultrasound imaging device configured to obtain one or more images of a catheter inserted into vasculature of a patient; and
a base unit configured to receive the images from the ultrasound imaging device when the ultrasound imaging device is positioned overtop a distal tip of the catheter that is inserted into the vasculature of the patient.

14. The imaging system of claim 13, further comprising:
a securement platform comprising a base layer, a vascular access device pocket formed in the base layer, a catheter insertion site window formed in the base layer distal to the vascular access device pocket, and an imaging device pocket, wherein
the base unit is configured to receive the images from the ultrasound imaging device positioned within the ultrasound imaging device pocket when the ultrasound imaging device pocket is positioned overtop the distal tip of the catheter that is inserted into the vasculature of the patient.

15. The imaging system of claim 13, wherein the base unit includes an artificial intelligence engine that is configured to detect a depth of the distal tip of the catheter from the images.

16. The imaging system of claim 13, further comprising:
a display for displaying the images, wherein the images comprise a transverse view of the catheter and a cross-sectional view of the catheter.

17. The imaging system of claim 14, wherein the securement platform comprises a first component that includes the vascular access device pocket and the catheter insertion site window and a second component that includes the imaging device pocket.

18. The imaging system of claim 14, further comprising:
a vascular access device having a stabilization platform, the vascular access device pocket being configured to receive the stabilization platform.

19. A method for obtaining images of a patient's vasculature comprising:
positioning an ultrasound imaging device to obtain one or more images of a catheter inserted into vasculature of a patient; and
displaying the one or more images of the catheter to allow a clinician to see the catheter inserted into the vasculature of the patient.

20. The method of claim 19, further comprising positioning a securement platform on the patient, the securement platform comprising an imaging device pocket that is positioned overtop the catheter that is inserted into the vasculature of the patient,
wherein the securement platform further comprises a base layer, a vascular access device pocket formed in the base layer, and a catheter insertion site window formed in the base layer distal to the vascular access device pocket.
